(19) United States
(12) Patent Application Publication
(10) Pub. No.: US 2014/0093926 A1
(43) Pub. Date: Apr. 3, 2014

(54) INTEGRATED SYSTEMS AND METHODS FOR ORGANIC ACID PRODUCTION

(71) Applicants: BOARD OF TRUSTEES OF MICHIGAN STATE UNIVERSITY, East Lansing, MI (US); THE MICHIGAN BIOTECHNOLOGY INSTITUTE, Lansing, MI (US)

(72) Inventors: Robert Hanchar, Charlotte, MI (US); Michael Guettler, Holt, MI (US); Venkataraman (Bobby) Bringi, Haslett, MI (US); Sanchin Jadhav, Lansing, MI (US); Dennis Miller, Okemos, MI (US); Lars Peereboom, Haslett, MI (US); Aspi K. Kolah, Mason, MI (US)

(73) Assignees: BOARD OF TRUSTEES OF MICHIGAN STATE UNIVERSITY, East Lansing, MI (US); THE MICHIGAN BIOTECHNOLOGY INSTITUTE, Lansing, MI (US)

(21) Appl. No.: 14/044,748

(22) Filed: Oct. 2, 2013

Related U.S. Application Data
(60) Provisional application No. 61/709,036, filed on Oct. 2, 2012.

(51) Int. Cl. C12M 1/00; C12P 7/46

(52) U.S. Cl. CPC C12M 43/00; C12P 7/46
USPC 435/145; 435/289.1

(57) ABSTRACT

Crude bio-based organic acid-producing feedstock is used to produce a bio-based organic acid. Related systems and methods are also described such as an integrated method including a step of producing a crude polyol product and then processing the crude polyol product to produce a bio-based organic acid.

### TECHNICAL FIELD OF THE INVENTION

The invention relates to systems and methods for producing organic acids, particularly bio-based organic acids.

### BACKGROUND

Bio-based organic acids have potential for displacing petrochemically derived monomers in a range of industrial applications such as polymers, food, pharmaceuticals and cosmetics. For example, organic acids, such as dicarboxylic acids, are useful in a number of processes, such as in the production of copolymers including but not limited to polyamides and polyesters. The use of bio-based organic acids produced from renewable energy sources can advantageously decrease reliance on fossil fuels and benefit the environment in terms of reduced carbon dioxide production.

Bio-based organic acids can be produced from a variety of carbon sources. However, attempts to produce bio-based organic acids, instead of petrochemically derived organic acids, have suffered from various limitations. For example, the economics of an organic acid-producing fermentation process are dependent on a number of factors including but not limited to feedstock cost, conversion yield, productivity and fermentation performance. Optimizing one such factor has often been at the expense of another. For example, although glucose is a common feedstock for such a process, it is known that using a polyol carbon source such as sorbitol as the feedstock can significantly increase the conversion yield relative to glucose. However, polyol feedstock materials are very costly relative to other typical carbon sources for bio-based organic acid production such as glucose, particularly because the polyol are significantly processed, e.g., crystallized, concentrated, and/or collected, to produce a concentrated purified product, e.g., syrup or crystalline form, that can be collected in a form suitable for storage and transportation prior to use in the fermentation process.

U.S. Patent Application Publication No. 2012/0151827 discloses a biomass conversion system for processing cellulosic biomass into biofuel comprising two reduction reactors in series and in fluid communication with one another.

### BRIEF DESCRIPTION OF THE DRAWINGS

FIG. 1 is a flow diagram of an integrated carbon source production and bio-based organic production process according to various embodiments of the invention.

FIG. 2 is a flow diagram of an integrated crude sorbitol and bio-based succinic acid production process according to various embodiments of the invention.

FIG. 3. is a flow diagram of an integrated carbon source production and bio-based organic production process incorporating an optional filtration component to remove solids from the crude bio-based organic acid-producing feedstock and an optional column capable of binding residual metals and/or metal ions to remove metals and/or metals ions from the crude bio-based organic acid-producing feedstock prior to its introduction into the second apparatus.

FIG. 4 is a graph showing production of succinic acid from a glucose carbon source.

FIG. 5 is a graph showing production of succinic acid from a commercial sorbitol source.

### DETAILED DESCRIPTION

In the following detailed description, embodiments are described in sufficient detail to enable those skilled in the art to practice them, and it is to be understood that other embodiments may be utilized and that chemical and processing changes may be made without departing from the spirit and scope of the present subject matter. The following detailed description is, therefore, not to be taken in a limiting sense, and the scope of the present invention is defined only by the appended claims.

In the various embodiments provided herein, efficient and economical methods and systems for producing bio-based organic acids are provided. In particular, the embodiments described herein include using a crude polyol feedstock in a process for producing organic acids. The crude polyol feedstock advantageously can be produced ‘on site’ from a bio-based carbon source-producing feedstock in an integrated process using an integrated system in which a first apparatus for producing the crude polyol feedstock product has an outlet in fluid communication with an inlet of a second apparatus for producing the bio-based organic acid from the crude polyol feedstock product. The use of the crude polyol product as the feedstock in a process for producing organic acids, particularly when combined with an optional step of producing the crude polyol feedstock product on site in an integrated process, advantageously allows bio-based organic acids to be produced in fewer steps and in a cost effective manner enabling the utilization of crude polyol feedstock products, such as crude sorbitol, in a fermentation process for producing succinic acid. Surprisingly, it has been advantageously found that significant processing of the crude polyol product, for example, by collecting, crystallizing, and/or concentrating the desired polyol, to produce a concentrated purified product (e.g., syrup or crystalline form) suitable for storage and transportation, is not required prior to fermentation.

An integrated system for preparing a bio-based organic acid is also described. The integrated system comprises a first apparatus for producing a crude polyol feedstock product and a second apparatus for producing a bio-based organic acid from the crude polyol feedstock product, wherein the second apparatus has an inlet in fluid communication with an outlet of the first apparatus such that at least a portion of the crude polyol feedstock product produced in the first apparatus can be easily and inexpensively transferred to the second apparatus for producing a bio-based organic acid, without need for concentration, collection, or crystallization of the crude polyol feedstock product. The integrated system described herein therefore advantageously enables the conversion in the first apparatus of an inexpensive carbon source-producing feedstock such as hydrolyzed biomass to a crude polyol feedstock product such as sorbitol that itself can then be transferred via fluid communication between the first apparatus and the second apparatus to the second apparatus such as a fermentor. As a result, bio-based organic acids are produced in fewer steps and in a cost effective manner.

The integrated biological processes described herein save time and expense by enabling the utilization of a significantly non-purified feedstock for bio-based organic acid production. The methods are also economical since a number of steps can now be eliminated and polyol feedstocks such as sorbitol that were previously cost-prohibitive can now advantageously be exploited. Furthermore, fermentation of the crude bio-based organic acid-producing feedstock with a suitable microorganism biocatalyst produces bio-based organic acids at rates and yields comparable or better than conventional methods obtained with commercial or refined polyols.

In one representative embodiment, glucose is hydrogenated in the first apparatus (i.e., the first apparatus is a hydrogenation reactor comprising a hydrogenation catalyst) to produce crude sorbitol, without any concentration, collection, or crystallization of the crude polyol product, and the crude polyol product is then transferred via fluid communication to a second apparatus comprising a fermentor and used as the feedstock in a fermentation process to produce succinic acid efficiently and economically. Surprisingly, crude sorbitol can be used for production of bio-based organic acids in a process which is at least as good, if not better, than commercial sorbitol. As mentioned previously, commercial sorbitol is costly and has been significantly purified by concentration, collection, and/or crystallization. Thus, commercial sorbitol is too costly to use in the commercial production of organic acids and has a different composition than the crude sorbitol product that serves as the crude bio-based organic acid-producing feedstock in the integrated process described herein.

In the various embodiments described herein, the use of crystallization, collection, and/or concentration steps can be eliminated in the preparation of a crude feedstock for use in producing bio-based organic acids, resulting in a significant cost savings relative to commercial sorbitol. Surprisingly, a crude sorbitol product can be used for production of bio-based organic acids in a process which is at least as good, if not better, than commercial sorbitol. Unexpectedly, the presence of significant contaminants in the crude sorbitol feedstock do not adversely impact the organic acid fermentation conversion yield. In particular, it was expected that residual catalyst contaminants would deleteriously affect the conversion yield of sorbitol to organic acid because such catalysts can negatively affect the microorganisms used in fermentations and thus the fermentation performance.

In one embodiment, minimal purification (e.g., filtration to remove solids) of the crude bio-based organic acid-producing feedstock (e.g., crude sorbitol) is performed as part of the integrated process for the production of bio-based organic acids. In another embodiment, the crude bio-based organic acid-producing feedstock is minimally purified as part of the integrated process using a column containing a material capable of binding residual metals and/or metal ions to remove metals and/or metals ions from the crude bio-based organic acid-producing feedstock prior to its introduction into the second apparatus. The filtration component and/or the column, when present, can be disposed between the first apparatus and the second apparatus. Thus, for example, the first apparatus may have an outlet in fluid communication with an inlet of the filtration component (when present) and the filtration component may have an outlet in fluid communication with an inlet of the second apparatus. Similarly, the first apparatus may have an outlet in fluid communication with an inlet of the column capable of binding residual metals and/or metal ions (when present) and the column may have an outlet in fluid communication with an inlet of the second apparatus. Alternatively, the first apparatus may have an outlet in fluid communication with an inlet of the filtration component (when present) and the filtration component may have an outlet in fluid communication with an inlet of the column (when present) and the column may have an outlet in fluid communication with an inlet of the second apparatus. The relative order of the filtration component and the column is immaterial provided that the first apparatus and the second apparatus are in fluid communication with one another via the optional filtration and/or components mentioned above. Of course, there need not be any intervening components between the first apparatus and the second apparatus such that the first apparatus and the second apparatus are in direct fluid communication with one another.

The term “betaine” as used herein typically refers to the free base N, N, N, trimethylglycine or glycine betaine which has the formula (CH₃)₃N⁺CH₂CO₂H. The term betaine may also refer to the neutral zwitterion form of betaine and/or an addition salt thereof such as betaine-HCl.

The term “biomass” as used herein, refers in general to organic matter harvested or collected from a renewable biological resource as a source of energy. The renewable biological resource can include plant materials, animal materials, and/or materials produced biologically. The term “biomass” does not include fossil fuels, which are not renewable.

The term “plant biomass” or “ligno-cellulosic biomass” (LCB) as used herein refers to virtually any plant-derived organic matter containing cellulose and/or hemicellulose as its primary carbohydrates (woody or non-woody) available for producing energy on a renewable basis. When used without a qualifier, the term “biomass” is intended to refer to LCB.

The term “hydrolyzed biomass” as used herein refers to hydrolyzed solids, such as hydrolyzed polysaccharides, which can include, for example, monomeric sugars (e.g., glucose, xylose, arabinose, mannose and/or galactose), disaccharides (e.g., sucrose, lactose, trehalose and/or maltose) and/or oligomeric sugars such as gluco-oligomers (e.g., linear chains of glucose units with varying degrees of polymerization (DP) and/or xylo-oligomers (e.g., xylose backbone polysaccharides with varying DP). Hydrolyzed biomass is one example of a carbon source-producing feedstock useful herein.

The term “carbon source-producing feedstock” as used herein refers to a bio-based feedstock capable of producing a carbon source.

The term “carbon source” as used herein refers to a carbon-containing gas, liquid or solid useful as a bio-based feedstock for producing bio-based organic acids.

The term “polyol” as used herein, refers to a sugar alcohol which can be used as a carbon-source producing feedstock. As such, a sugar alcohol is a hydrogenated form of a carbohydrate whose carbonyl group (aldehyde or ketone, reducing sugar) has been reduced to a primary or secondary hydroxyl group. Simple sugar alcohols have the general formula H(HCHO)ₙ₊₁H, wherein typically n is between 1 and 6, more often either 4 or 5.

The terms “refined polyol” or “commercial polyol” refer to commercial grade or “purified polyol” that may be produced by subjecting a crude polyol product to art-recognized purification processes involving concentration, crystallization, and/or collection. Such purification steps are typically conducted to remove contaminants (e.g., impurities, and the like) and to provide the polyol in a more stable form suitable for storage and/or transport. Refined polyol is defined herein as being at least 98.5% pure, i.e., a composition comprising refined polyol comprises at least 98.5 weight % of the specific polyol.

The term “crude polyol product” as used herein refers to a polyol product produced according to various known processes (e.g., typically, fermentation or hydrogenation) that is not further subjected to art-recognized purification processes such as concentration, crystallization, and/or collection. As such, crude polyols have a purity less than that of refined polyol.

The term “bio-based organic acid” as used herein refers to an organic acid produced from a renewable energy source. Bio-based dicarboxylic acids (e.g., succinic acid) are one type of bio-based organic acid. When used without further qualification herein, the terms organic acid, dicarboxylic acid, succinic acid, and the like, refer to bio-based organic acids.

The term “fermentation media” as used herein describes the media for growing suitable microorganisms and conducting a fermentation (prior to inoculation with the microorganism). Similarly, the term “fermentation broth” refers to the fermentation media after inoculation, i.e., after fermentation has been initiated.

As used herein, the term “integrated” refers to a process in which a series of steps are performed in combination to form the entire integrated process, particularly such that there is no offline processing subsequent to any intermediate step of the integrated process. Similarly, the term “integrated” refers to a system having apparatus components that are operably connected to one another in series so as to avoid the need for any offline processing prior to production of the final desired product.

“Offline processing” as used herein refers to processing steps conducted with components that are not directly connected to the first apparatus or the second apparatus such that transfer from the first apparatus to the second apparatus cannot occur by fluid communication between the first apparatus and second apparatus. Offline processing therefore does not occur when a processing component is disposed between the first apparatus and the second apparatus provided that the first apparatus and the second apparatus are in fluid communication with one another via the component(s).

The methods described herein provide for an economical bio-based method for producing organic acids from crude polyols, such as crude sorbitol. In one embodiment, an integrated method is provided comprising processing, in a first apparatus, a carbon source-producing feedstock to produce a crude polyol product and processing, in a second apparatus, the crude polyol product to produce a bio-based organic acid, wherein the second apparatus has an inlet in fluid communication with an outlet of the first apparatus. The second apparatus may be in direct fluid communication with the first apparatus or an optional processing component may be disposed between the first apparatus and the second apparatus such that the second apparatus and the first apparatus are in fluid communication. Thus, an integrated system for preparing a bio-based organic acid according to the disclosure comprises a first apparatus for producing a crude polyol product, and a second apparatus for producing a bio-based organic acid from the crude polyol product, wherein the second apparatus has an inlet in fluid communication with an outlet of the first apparatus.

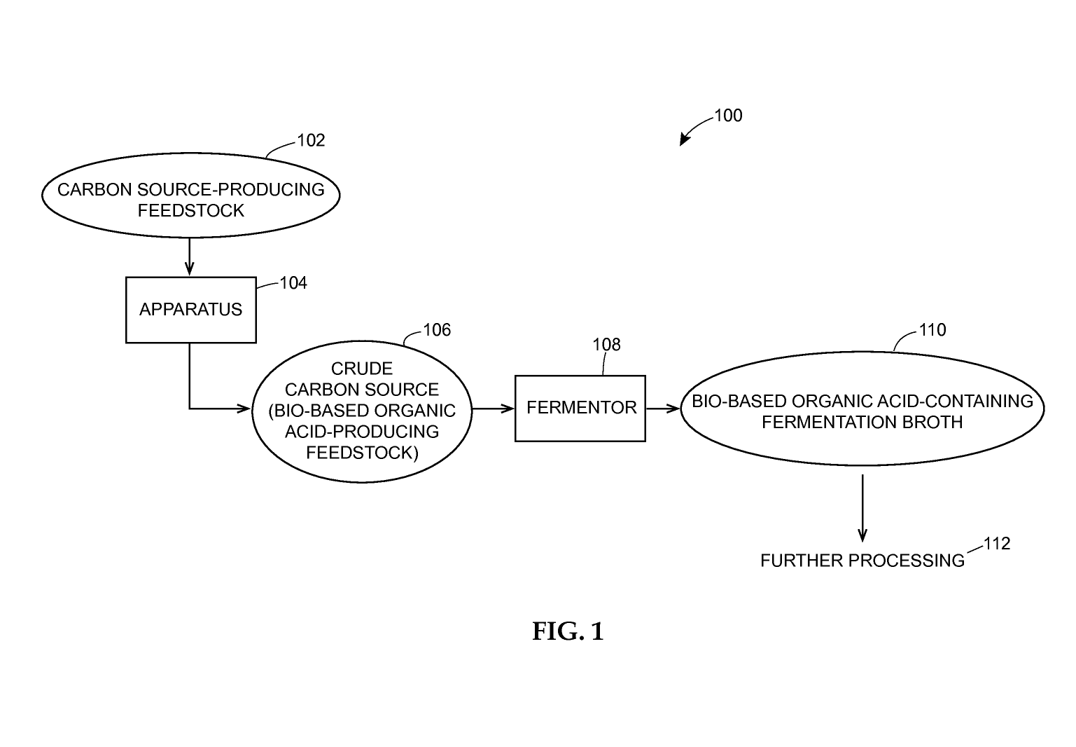

In the embodiment shown in FIG. 1, an integrated process 100 is provided in which a carbon source-producing feedstock 102 is provided to an inlet of an apparatus 104 to produce a crude polyol 106, which also serves as a bio-based organic acid producing feedstock as shown. As part of the integrated process 100, the crude polyol 106 is provided from an outlet of the apparatus 104 to an inlet of a fermentor 108 to produce a bio-based organic acid-containing fermentation broth 110 which can optionally be subjected to further processing 112, such as to produce purified bio-based organic acid. Further processing can include biomass removal, concentration, acidification, crystallization, and collection. Recovery of organic acids is well-established and suitable recovery methods are disclosed, for example, in U.S. Pat. Nos. 6,265,190, 7,667,068, 6,284,904 and 5,034,105.

Any suitable carbon source-producing feedstock 102 can be used. For example, plant biomass, hydrolyzed biomass, starches, monomeric sugars, oligomeric sugars, and combinations of the foregoing may be used as the carbon source-producing feedstock. In one embodiment, a sugar carbon source is used, e.g., any type of monomeric sugar. In one embodiment, the carbon source-producing feedstock 102 is glucose. In another embodiment, the carbon source-producing feedstock 102 is hydrolyzed biomass.

Any suitable type of apparatus 104 can be used to produce the crude carbon source 106, including, but not limited to, a reactor, such as a hydrogenation reactor or a fermentor. The apparatus 104 used is dependent on the method for producing the crude carbon source 106. As such, any suitable method can be used to convert the carbon source-producing feed stock 102 to a crude carbon source 106, typically a crude polyol, including any known methods. In one embodiment, hydrogenation of glucose as the carbon-source producing feed stock 102 produces crude sorbitol as the crude carbon source 106 according to methods known in the art. In one embodiment, a hydrogenation catalyst comprises a nickel based catalyst (e.g., Raney nickel) or a ruthenium catalyst, and the temperature, pressure and reaction times are adjusted according to the specific type and loading of catalyst, as is known in the art.

In various embodiments, the temperature in the apparatus 104 may range from about 10° C. to about 200° C., pressure from about 0 to about 2500 psig, reaction time ranges from about 30 min to 100 hours with a catalyst loading of about 1% to about 50% wt/wt. of all reaction components in the apparatus 104.

In another embodiment, the crude carbon source 106 is produced by fermentation, such as with a microorganism capable of producing a desired crude polyol product. In this respect, U.S. Pat. No. 7,358,072, entitled “Fermentative Production of Mannitol” discloses suitable microorganisms for producing a desired crude polyol product from the carbon source-producing feedstock starting material 102.

In one embodiment, the crude polyol product 106 produced by hydrogenation or fermentation in apparatus 104 may include a variety of alcohols, such as sugar alcohols, including, but not limited to, methanol, glycol, glycerol, erythritol, threitol, arabitol, xylitol, ribitol, mannitol, sorbitol, galactitol, iditol, inositol, volemitol, isomalt, maltitol, lactitol, polyglycitol, and combinations thereof. In one embodiment, mixtures of sugar alcohols are produced from fermentation of lingo-cellulosic biomass.

In one embodiment, the carbon source-producing feedstock 102 is a non-purified glucose solution which is converted to a crude sorbitol product in a reduction reaction conducted in apparatus 104 and used without significant further processing (e.g., purification, concentration, and/or crystallization steps to significantly purify and/or isolate the sorbitol) as feedstock for a bio-based organic acid fermentation conducted in second apparatus 108 which is a fermentor.

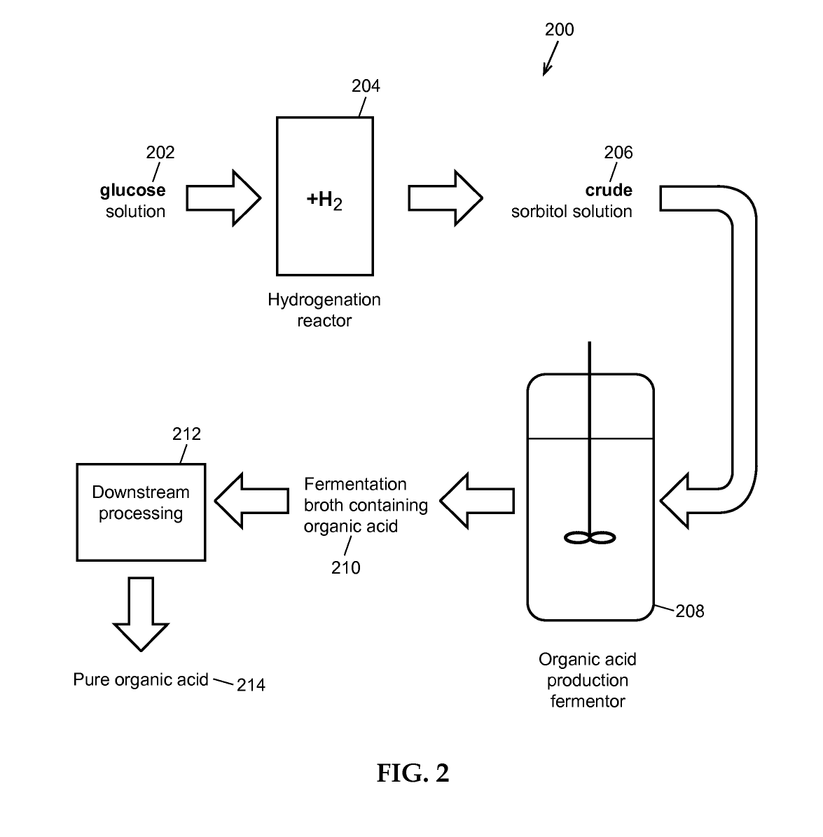

In the embodiment shown in FIG. 2, an integrated process 200 is provided in which the carbon source-producing feedstock is a glucose solution 202. Glucose solution 202 is provided to a hydrogenation reactor 204 in which the glucose solution 202 is reduced to provide a crude sorbitol solution 206 which does not undergo significant further processing (e.g., condensation, concentration, and/or crystallization steps to significantly purify and/or isolate the sorbitol) prior to its introduction into the second apparatus 208.

In one embodiment, the glucose solution 202 before reduction and the crude sorbitol solution 206 after reduction contain comparable amounts of glucose and sorbitol, respectively. In one embodiment, the glucose solution 202 prior to reduction is a solution containing about 10% to about 30% glucose, by volume. In this embodiment, the glucose solution 202 is reduced to provide a crude sorbitol solution product 206 containing about 10% to about 30% sorbitol, by volume, after reduction. Use of a crude sorbitol product rather than a refined sorbitol significantly reduces overall costs and thus makes the use of sorbitol an attractive alternative in the manufacture of a bio-based organic acid. In one embodiment, the conversion rate of glucose to sorbitol is less than 100%, but the disclosed methods are still beneficial in view of the better results obtained with sorbitol conversion relative to glucose conversion as described in Example 1.

Referring again to the embodiment shown in FIG. 2, the crude sorbitol 206 is provided to a fermentor 208 where a succinic acid-containing fermentation broth 210 is produced. The broth 210 can then be subjected to further processing 212 to produce a purified bio-based organic acid (e.g. succinic acid) 214. As mentioned previously, further processing can include biomass removal, concentration, acidification, crystallization, and collection.

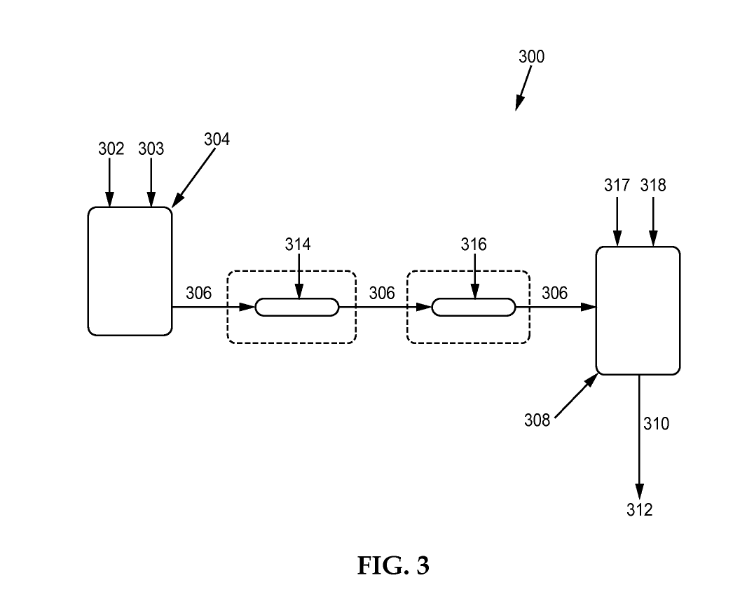

Turning now to the embodiment shown in FIG. 3, an integrated process 300 is provided in which a carbon source-producing feedstock 302 is provided to an inlet of an apparatus 304 to produce a crude polyol 306, which serves as a bio-based organic acid producing feedstock as shown. Any suitable type of apparatus 304 can be used to produce the crude carbon source 106, including, but not limited to, a reactor, such as a hydrogenation reactor or a fermentor. The apparatus 304 used is dependent on the method for producing the crude carbon source 306. As such, any suitable method can be used to convert the carbon source-producing feed stock 302 to a crude carbon source 306, typically a crude polyol, including any known methods as described above with respect to FIG. 1. When the apparatus 304 comprises a hydrogenation reactor, a hydrogenation catalyst such as a nickel based catalyst (e.g., Raney nickel) or a ruthenium catalyst is disposed within the reactor and hydrogen gas 303 is flowed into the reactor 304.

As part of the integrated process 300, the crude polyol 306 is provided from an outlet of the apparatus 304 to an inlet of a filtration component 314 to remove solids from the crude polyol product and then from an outlet of the filtration component into an inlet of a column 316 capable of binding residual metals and/or metal ions to remove metals and/or metals ions from the crude polyol product 306 prior to its introduction into the second apparatus 308. The second apparatus is a fermentor to which fermentation media 317 and a suitable microorganism biocatalyst are added such that the crude polyol can be fermented to produce a bio-based organic acid-containing fermentation broth 310 which can optionally be subjected to further processing 312, such as to produce purified bio-based organic acid.

As in the other embodiments, any suitable carbon source-producing feedstock 302 can be used.

As shown in FIG. 3, apparatus 304 can be a continuous flow hydrogenation reactor in which a solution of carbon source-producing feedstock 302 and hydrogen gas 303 are passed through a heated column of the hydrogenation reactor, the column of the hydrogenation reactor containing a hydrogenation catalyst. The crude polyol product from the first reactor 304 flows to the second reactor 308, optionally through filtration component 314 and/or metal binding column 316. Advantageously, the temperature, pressure, and residence time in the first reactor 304 can be selected to sterilize the feed solution 302 as it is hydrogenated, thereby providing a sterile crude polyol solution to the second reactor 308, and beneficially avoiding the need of a separate continuous sterilization apparatus.

Crude sorbitol for use herein may be produced by various processes known in the art. In one embodiment, crude sorbitol is produced by hydrogenation of fructose, glucose or mixtures thereof in aqueous solution at high temperature in the presence of a hydrogenation catalyst, such as Raney nickel or a ruthenium catalyst.

In some embodiments, microorganisms (e.g., bacteria, fungi or yeast) may be grown in fermentation media containing crude sorbitol as a crude carbon source to produce an organic acid. Exemplary organic acid end products include pyruvic acid, succinic acid, fumaric acid, malic acid, maleic acid, citric acid, propionic acid and combinations thereof.

Surprisingly, crude sorbitol can be used for production of bio-based organic acids in a process which is at least as good, if not better, than commercial sorbitol. In contrast to the inventors' expectations, organic acid fermentation is not significantly detrimentally impacted even when substantially all of the contaminants contained therein remain in the bio-based organic acid-producing feedstock. In one embodiment, the crude sorbitol product is green in color at the beginning of the fermentation and remains green in color throughout the fermentation process; the green color is consistent with residual catalyst being present in the crude sorbitol product. The crude sorbitol product may contain more than 10 ppm catalyst, more than 20 ppm catalyst or even more than 30 ppm catalyst. The crude sorbitol product may contain more than 10 ppm nickel, more than 20 ppm nickel or even more than 30 ppm nickel. In one embodiment, the chemical contaminants include, but are not limited to catalyst, sugars, metals (e.g. lead, aluminum), salts (e.g. sodium) and unconverted carbon source-producing feedstock.

In one embodiment, the integrated process described herein advantageously results in a cost reduction for the production of organic acids.

In one embodiment, a process for using a crude polyol product to produce a bio-based organic acid by a suitable microbial system is provided. In one embodiment, a crude sorbitol product is used with any suitable microbial system. In one embodiment, the microbial system is a prokaryote or a eukaryote and the bio-based organic acid is bio-based succinic acid.

In some embodiments, microorganisms may be grown in compositions containing crude sorbitol product as a carbon source to produce an organic acid. Exemplary organic acid include pyruvic acid, succinic acid, fumaric acid, malic acid, maleic acid, citric acid, propionic acid and combinations thereof.

The microorganisms used are not particularly limited so long as they have the ability to produce the organic acid of interest. Exemplary bacteria include members of the members of Pasteurellaceae family (e.g., Mannheimia ruminalis, members of the Actinobacillus genus including A. succinogenes; Bisgaard Taxon 6; Bisgaard Taxon 10; Mannheimia succiniciproducens; and Basfia succiniciproducens); E. coli; Anaerobiospirillum succiniciproducens; Ruminobacter amylophilus; Succinivibrio dextrinosolvens; Prevotella ruminicola; Ralstonia eutropha; members of the Coryneform genus including Corynebacterium glutamicum and Corynebacterium ammoniagenes; Brevibacterium flavum, Brevibacterium lactofermentum, Brevibacterium divaricatum; members of the Lactobacillus genus); yeast (e.g., members of the Saccharomyces genus); and any subset thereof. In some embodiments, recombinant variations of the microorganism may be used. In some embodiments, microorganisms that may be used may overexpress glucose-6 phosphate dehydrogenase enzyme, malate dehydrogenase enzyme or both.

In one embodiment, the microbial system is a member of the Pasteurellaceae family. In one embodiment, the microbial system comprises Actinobacillus succinogenes. In one embodiment, the microbial system can include the strains described in U.S. Patent Application Ser. No. 61/708,998, entitled “RECOMBINANT MICROORGANISMS FOR PRODUCING ORGANIC ACIDS” filed by the present applicants on Oct. 2, 2012, which application is incorporated by reference herein in its entirety. In other embodiments, the microorganisms include those described in U.S. Pat. No. 8,119,377, which patent is incorporated by reference herein in its entirety.

The methods for producing an organic acid can include growing suitable microorganisms in a suitable fermentation media which contains a crude carbon source (e.g., a bio-based organic acid-producing feedstock such as crude sorbitol). In one embodiment, the fermentation media also contains a nitrogen source, inorganic salts, vitamins or growth promoting factors, and the like. In some embodiments, the salts, ammonium source and other nutrient media requirements may be obtained from corn steep liquor (CSL), a by-product of the corn wet-milling industry.

Fermentations can be conducted by combining the crude carbon source and fermentation media in any suitable fermentor, and inoculating with a suitable microorganism. Fermentation may be carried out either aerobically or anaerobically under conditions conducive to the growth of the microorganism and production of the suitable organic acid. In one embodiment, the fermentation temperature is maintained within the range of at least about 25° C. and less than about 50° C. In some embodiments, the temperature is between about 30° C. and about 40° C.

In one embodiment, the pH of the fermentation media at the beginning of fermentation is within the range of about 6-7, and can be controlled by addition of base to maintain the pH between about pH 4.5 to about 8 or between about 5 to about 7.2 as the fermentation progresses. Mg(OH)₂, MgCO₃, NH₄OH, NaOH and/or gaseous NH₃, may be used to control pH. In some embodiments, sodium carbonate can additionally or alternatively be used as a pH control agent.

The sodium concentration can range between about 1200 mg/l to about 6800 mg/L. In some embodiments, the sodium concentration is from about 3000 mg/l to about 3500 mg/L. Na₂CO₃, Na₃PO₄, Na₂HPO₄, NaH₂PO₄, NaHCO₃, NaCl, NaOH and mixtures thereof can be used to provide sodium to the fermentation media. Na from organic salts (e.g. monosodium glutamate, sodium acetate, and the like) may also be used.

The fermentation media may also include betaine. The betaine can be present as betaine-HCl, betaine free base, betaine zwitterions, or mixtures thereof.

In other embodiments, the betaine may be present in the fermentation media as a component of a feed product which contains betaine. Exemplary betaines or at least one feed product which contains betaine include betaine, amino acid fermentation byproduct solubles, molasses containing betaine, condensed separator byproduct, condensed molasses solubles, vinasse, or any mixture thereof. Other examples of feed products which contain betaine include a condensed, extracted glutamic acid fermentation product, amino acid fermentation byproduct solubles from the fermentative production lysine, amino acid fermentation byproduct solubles from the fermentative production threonine, or amino acid fermentation byproduct solubles from the fermentative production tryptophan. The betaine concentration may range from about 0.05 g/l to about 2 g/l. In some embodiments, the betaine concentration used in the initial fermentation media may be about 0.2 g/l to about 0.5 g/l.

Embodiments will be further described by reference to the following examples, which are offered to further illustrate various embodiments of the present subject matter. It should be understood, however, that many variations and modifications may be made while remaining within the scope of the present subject matter

### Example 1

### Comparison of Glucose and Commercial Sorbitol in Fermentation of Bio-Based Succinic Acid

Actinobacillus succinogenes (FZ53/p856.78) as described in Application Ser. No. 61/708,998, entitled “RECOMBINANT MICROORGANISMS FOR PRODUCING ORGANIC ACID” was cultivated using a Bioflo III fermentor (New Brunswick) containing 2 L of culture medium. The culture medium contained 120 g/L carbon source; glucose (Cerelose™, Industrial Commodities Inc.) or sorbitol (Crystalline, NF/FCC, ADM Co.), 30 g/L corn steep liquor (“CSL”) (10-12% solids, ADM Co.), 1.6 g/L Mg(OH)₂, 0.2 mg/L biotin, 0.5 g/L betaine HCl, 0.2 g/L monosodium glutamate (“MSG”), 6.5 mM sodium phosphate, 7 g/L Na₂CO₃, and 0.5 g/L yeast extract (AG900). All chemicals were obtained from Sigma and were reagent grade (unless specified otherwise).

The fermentor was inoculated with 6.25% (v/v) inoculum (containing FZ53/p856.78) from a vial culture cultivated in the same medium as the fermentor and incubated with constant shaking at 150 rpm at 38° C. for 13 h. The inoculated fermentor was incubated at 38° C. with agitation at 380 rev/min and a sparge of 0.05 volume of sparger gas per reactor volume per minute (vvm) CO₂. The pH of the fermentation medium was maintained at about 6.8 by automatic addition of 6 M Mg(OH)₂.

The carbon source feed was implemented between 12 and 22 h during which 15 g of additional carbon source (sorbitol or glucose) was added to the fermentor.

### Residual Sugar and Bio-Based Organic Acid Determination

Residual sugar and bio-based organic acid concentrations in the culture supernatants and filtrates were determined by HPLC (Agilent 1200 series). An Aminex HPX-87H (300 mm×7.8 mm) column (Bio-Rad) was used with a mobile phase consisting of 0.013 N H₂SO₄ with a flow rate of 1.4 ml/min. Analyte peaks were detected and quantified using a refractive index detector (Waters 2414). Peak identification was determined by reference to organic acid standard solutions purchased from Sigma.

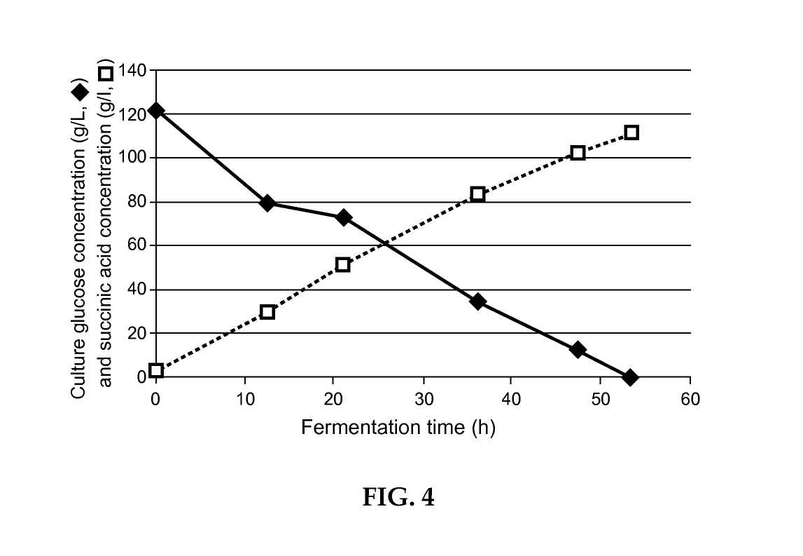

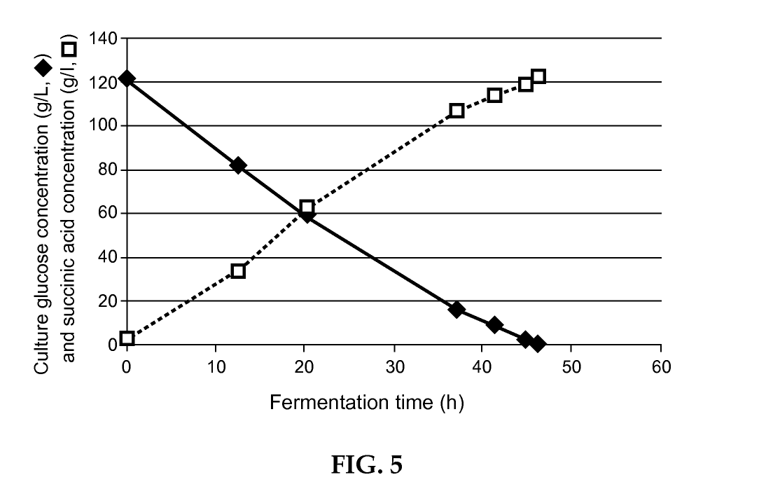

FIG. 4 shows graphically the results of production of bio-based succinic acid using glucose as a carbon source. FIG. 5 shows graphically the results of production of bio-based succinic acid using sorbitol as a carbon source. Table 1 below summarizes the results:

TABLE 1

| | | | | Yield (g succinic |
|---|---|---|---|---|
| Fermentation | | Productivity | Titer | acid/g carbon |
| Number | Carbon Source | (g/Lh) | (g/L) | source) |
| 809-12 | Glucose | 2 | 111.1 | 0.93 |
| 722-12 | Sorbitol | 2.6 | 121.4 | 1.17 |

As demonstrated by these data, fermentation performance using sorbitol as the carbon source showed better results than glucose in terms of fermentation parameters such as titer, productivity and yield. Thus, the utilization of a polyol such as sorbitol as the bio-based organic acid-producing feedstock in the manufacture of a bio-based organic acid is desirable.

### Example 2

### Production of Crude Sorbitol

A 2-liter Parr reactor equipped with a mechanical stirrer was charged with 450 grams of glucose (Sigma-Aldrich) dissolved in 1.5 liters of de-ionized water. To this was added 100 g of Raney nickel (Pressure Chemical Company). The vessel was sealed and pressurized with hydrogen gas. While maintaining the hydrogen gas pressure at between 800 and 700 psi, the reaction heated to 80° C. and stirred at 1000 rpm for 36 hours. The reaction mixture was allowed to cool to room temperature and the mixture was filtered to remove the solid Raney nickel catalyst. The resulting solution had a green tint and contained 262 g/L sorbitol and 4.5 g/L glucose. No attempt was made to crystallize and collect the sorbitol from the residual glucose or to remove the Raney nickel that had been solubilized during the reaction.

### Example 3

### Comparison of Commercial and Crude Sorbitol in Fermentation of Bio-Based Succinic Acid

### Starting Materials

Commercial sorbitol from the same source as Example 1 and crude sorbitol made according to the method described in Example 2 were used. Table 2 is a comparison of the appearance and contents of commercial and crude sorbitol used in this testing.

TABLE 2

| | Commercial Sorbitol | Crude Sorbitol | |
|---|---|---|---|
| Physical | White crystalline solid | Green transparent solution | |
| Appearance | | | |
| Sorbitol content | 91-105%* | 265 | g/L |
| Glucose content | ≦0.3%* | 4.7 | g/L (1.7%) |
| Nickel | ≦1 ppm* | 95.5 | ppm |
| Lead | ≦1 ppm* | <0.125 | ppm |
| Aluminum | Not Reported | 813 | ppm |
| Sodium | Not Reported | 363 | ppm |

*data from commercial product manufacturer's specification document

### Fermentation Conditions

Actinobacillus succinogenes (FZ53/p856.78) as described in Example 1 was cultivated using a Bioflo III fermentor (New Brunswick) containing 2 L of the same medium and under the same fermentation conditions provided in Example 1. Samples were periodically removed for determination of the residual carbon source (e.g., commercial or crude sorbitol) as indicated in Table 3.

### Analytical Methods

The residual amounts (e.g. the residual carbon source measurements indicate the amount of carbon source remaining in the medium and by subtraction the amount of carbon source utilized. The fermentations are stopped when the residual carbon source is 0 g/L as this means all the carbon source has been consumed) of commercial sorbitol and crude sorbitol, as well as the bio-based succinic acid concentration in the culture filtrates, were determined as described in Example 1.

Table 3 compares the results of bio-based succinic acid production under anaerobic conditions using commercial versus crude sorbitol.

TABLE 3
Results of Bio-Based Succinic Acid Production

| | | | | Yield* | Succinic |
|---|---|---|---|---|---|
| | Incubation | | | g/g | acid |
| | Time | Sorbitol | Succinic | carbon | productivity |
| | (h) | (g/L) | (g/L) | source | (g/Lh) |
| Commercial | 0 | 122.7 | 2.7 | | |
| Sorbitol | 20 | 62.1 | 59 | 1.08 | 2.8 |
| (802-12) | 48.1 | 0 | 121.6 | 1.15 | 2.5 |
| Crude Sorbitol | 0 | 122.2 | 3.1 | | |
| (801-12) | 20.2 | 63.9 | 60 | 1.14 | 2.8 |
| | 50.9 | 0 | 124.3 | 1.18 | 2.4 |

*CO₂ is incorporated into the final product such that the g/g carbon source yield can exceed 1; the maximum theoretical yield with glucose is 1.12 g/g and with sorbitol is 1.20 g/g.

This example demonstrates that crude sorbitol can be used as a bio-based organic acid-producing feedstock as it supported growth and bio-based succinic acid production in comparable amounts as compared with commercially obtained, crystalline sorbitol. As compared with commercial sorbitol, crude sorbitol can also be made through a much simpler process which eliminates steps, such as, forcing the sorbitol-producing reaction to run to completion and/or significantly purifying, collecting, crystallizing, and/or concentrating the resulting sorbitol. Elimination of such steps also allows the production of bio-based succinic acid to be much more economical. Further advantages are realized by integrating the feedstock production and fermentation reaction in the same facility as described herein.

### Example 4

### Growth of Mannheimia succiniciproducens on Crude Sorbitol

Various succinate producing organisms are grown using a culture medium containing crude sorbitol. Mannheimia succiniciproducens such as LPK7 are cultivated in a culture medium containing a nitrogen source and trace metals, for example, the MMH3 medium (as described by Young Hoon et al 2010, J. Microbiol. Biotechnol., 20, 1677-1680) and are grown on various carbon sources such as glucose (e.g., Cerelose™), refined sorbitol (available from ADM Inc.) or crude sorbitol (e.g., prepared as described in Example 2). The organism is cultivated as described in Jae et al 2009, J. Microbiol. Biotechnol., 19, 167-171.

The residual carbon source and the succinic acid produced are determined as described in Example 1. Consistent with the results shown in example 3, it is expected that organic acid will be produced at comparable levels when using crude sorbitol and refined sorbitol.

### Example 5

Basfia sp. is cultivated as described in Scholten & Dägele (2008, Biotechnol. Lett. 30, 2143-2146) except the carbon sources used will be glucose (e.g., Cerelose™) refined sorbitol (e.g., ADM Inc.) and crude sorbitol (e.g., prepared as described in Example 2).

The residual carbon source and the succinic acid produced are determined as described in Example 1. Consistent with the results shown in example 3, it is expected that organic acid will be produced at comparable levels when using crude sorbitol and refined sorbitol.

### Example 6

An E. coli strain genetically engineered to produce succinic acid, such as those described in Zhang et al (2009, PNAS 106, 21080-20185) is cultured under suitable culture conditions (such as those described in Zhang et al. 2009) with the carbon source being glucose (e.g., Cerelose™), refined sorbitol (e.g., available from ADM) or crude sorbitol (e.g. prepared as described in Example 2).

The residual carbon source and the succinic acid produced are determined as described in example 1. Consistent with the results shown in example 3, it is expected that organic acid will be produced at comparable levels when using crude sorbitol and refined sorbitol.

### Example 7

A strain of S. cerevisiae which has been metabolically engineered to produce succinic acid via the reductive TCA cycle (for example, by the heterologous expression or overexpression of phosphoenol pyruvate carboxykinase activity, malate dehydrogenase activity and fumarase activity) is cultivated in anaerobic bottles containing medium similar to that described by Zelle et al. (2010, Appl. Environ. Microbiol. 76, 744-750). The culture may be incubated aerobically initially to promote biomass generation before transferring to an anaerobic condition for succinic acid production or may be cultured anaerobically throughout. The culture medium is tested with glucose, commercial sorbitol and crude sorbitol (prepared as described in Example 2). The carbon source utilization and product formation are determined as described in example 1.

Consistent with the results shown in example 3, it is expected that organic acid will be produced at comparable levels when using crude sorbitol and refined (commercial) sorbitol.

The various embodiments provide for a method comprising producing a bio-based organic acid-containing fermentation broth from a non-purified carbon source and a bio-based catalyst (e.g., microorganism, such as from the Pasteurellaceae family).

In one embodiment, an integrated method is provided comprising producing a crude polyol; and using the crude polyol to produce a bio-based organic acid.

In one embodiment, the non-purified carbon source is crude polyol (e.g., unprocessed sorbitol and the bio-based organic acid produced in the fermentation broth is a dicarboxylic acid (e.g., bio-based succinic acid).

In one embodiment, the biocatalyst is grown in a fermentation media containing corn steep liquor, betaine, sodium, magnesium ions and combinations thereof. The bio-based catalyst may include, for example, Actinobacillus succinogenes, Basfia succiniciproducens, or Mannheimia succiniciproducens.

In one embodiment, the method further comprises producing the crude polyol from a carbon source-producing feedstock.

In one embodiment, a method is provided comprising using a crude polyol and/or polyol product to produce a bio-based organic acid with a microorganism (e.g., prokaryote or eukaryote). In one embodiment, the microorganism is from a Pasteurellaceae family, such as Actinobacillus succinogenes.

In one embodiment, a system is provided comprising a fermentor for producing a bio-based organic acid-containing fermentation broth from a crude polyol and a bio-based catalyst.

In one embodiment, an integrated system is provided comprising an apparatus (e.g., hydrogenator or fermentor) for producing a crude polyol; and a fermentor for producing a bio-based organic acid-containing fermentation broth from the crude polyol and a bio-based catalyst.

Although specific embodiments have been illustrated and described herein, it will be appreciated by those of ordinary skill in the art that any procedure that is calculated to achieve the same purpose may be substituted for the specific embodiments shown. This application is intended to cover any adaptations or variations of the present subject matter. For example, although the various embodiments have been described, it is understood that other processes and feedstocks may be used. Therefore, it is manifestly intended that embodiments be limited only by the claims and the equivalents thereof.

1. An integrated method for producing a bio-based organic acid comprising:
processing, in a first apparatus, a carbon source-producing feedstock to produce a crude polyol product; and
processing, in a second apparatus, the crude polyol product to produce a bio-based organic acid, wherein the second apparatus is a fermentor and has an inlet in fluid communication with an outlet of the first apparatus.

2. The method of claim 1, wherein the carbon source-producing feedstock comprises hydrolyzed biomass.

3. The method of claim 1, wherein the carbon source-producing feedstock is selected from the group consisting of monomeric sugars, oligomeric sugars, and mixtures thereof.

4. The method of claim 1, wherein the carbon source-producing feedstock comprises fructose, glucose, or mixtures thereof.

5. The method of claim 1, wherein the crude polyol product comprises a sugar alcohol.

6. The method of claim 1, wherein the crude polyol product comprises a sugar alcohol selected from the group consisting of methanol, glycol, glycerol, erythritol, threitol, arabitol, xylitol, ribitol, mannitol, sorbitol, galactiol, iditol, inositol, volemitol, isomalt, malitol, lactitol, polyglycitol, and combinations thereof.

7. The method of claim 1, wherein the crude polyol product comprises sorbitol.

8. The method of claim 1, wherein the bio-based organic acid comprises a dicarboxylic acid.

9. The method of claim 1, wherein the bio-based organic acid is selected from the group consisting of pyruvic acid, succinic acid, fumaric acid, malic acid, maleic acid, citric acid, propionic acid and combinations thereof.

10. The method of claim 1, wherein the first apparatus comprises a hydrogenation reactor.

11. The method of claim 1, wherein the crude polyol product comprises more than 10 ppm catalyst, more than 20 ppm catalyst, and/or more than 30 ppm catalyst.

12. The method of claim 1, wherein the crude polyol product comprises more than 10 ppm nickel, more than 20 ppm nickel, and/or more than 30 ppm nickel.

13. The method of claim 1, wherein the first apparatus comprises a hydrogenation reactor, the carbon source-producing feedstock comprises fructose, glucose, or mixtures thereof, the crude polyol product comprises sorbitol, and the bio-based organic acid comprises succinic acid.

14. The method of claim 1, wherein the second apparatus comprises a fermentation broth and a microorganism.

15. The method of claim 14, wherein the microorganism is a prokaryote or a eukaryote.

16. The method of claim 14, wherein microorganism is a member of the bacterial family Pasteurellaceae.

17. The method of claim 14, wherein the microorganism comprises Actinobacillus succinogenes, Basfia succiniciproducens, or Mannheimia succiniciproducens.

18. An integrated system for preparing a bio-based organic acid comprising:
a first apparatus for producing a crude polyol product; and
a second apparatus for producing a bio-based organic acid from the crude polyol product, wherein the second apparatus is a fermentor and has an inlet in fluid communication with an outlet of the first apparatus.

19. The integrated system of claim 18, wherein the first apparatus comprises a hydrogenator or a fermentor.

20. The integrated system of claim 18, wherein the first apparatus comprises a hydrogenation reactor.

21. The integrated system of claim 20, wherein first apparatus includes a carbon source-producing feedstock and a hydrogenation catalyst.

22. The integrated system of claim 21, wherein the carbon source-producing feedstock comprises fructose, glucose, or mixtures thereof.

23. The integrated system of claim 18, wherein the crude polyol product comprises a sugar alcohol and the second apparatus contains the crude polyol product.

24. The integrated system of claim 23, wherein the crude polyol product comprises sorbitol.

25. The integrated system of claim 18, wherein the second apparatus comprises a fermentor including a fermentation broth and a microorganism.
